(19) United States
(12) Patent Application Publication
(10) Pub. No.: US 2008/0312670 A1
(43) Pub. Date: Dec. 18, 2008

(54) Surgical ligature clip

(75) Inventors: Theodor Lutze, Balgheim (DE); Manfred Dworschak, Duerbheim (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/215,050

(22) Filed: Jun. 23, 2008

Related U.S. Application Data
(63) Continuation of application No. PCT/EP2006/006781, filed on Jul. 11, 2006.

(30) Foreign Application Priority Data
Jan. 11, 2006 (DE) 20 2006 000 329

(51) Int. Cl. A61B 17/122

(52) U.S. Cl. 606/157

(57) ABSTRACT

In order, in the case of a surgical ligature clip comprising two retaining arms, which are connected to one another in each case at one end by a deformable connection point and bendable in such a way towards one another that the arms move from an open position, in which they are spaced further apart from one another, into a closed position, in which the mutually opposing inner sides of the arms are brought permanently closer to one another, to improve the seat of the ligature clip on a hollow organ, it is proposed that it comprises an endless web member, which in the region of the two arms and the connection point forms two juxtaposed portions that merge one into the other at the free ends of the arms remote from the connection point.

This application is a continuation of international application number PCT/EP 2006/006781 filed on Jul. 11, 2006.

The present disclosure relates to the subject matter disclosed in international application PCT/EP 2006/006781 of Jul. 11, 2006 and German application number 20 2006 000 329.0 of Jan. 11, 2006, which is incorporated herein by reference in its entirety and for all purposes.

### BACKGROUND OF THE INVENTION

The invention relates to a surgical ligature clip comprising two retaining arms, which are connected to one another in each case at one end by a deformable connection point and bendable in such a way towards one another that the arms move from an open position, in which they are spaced further apart from one another, into a closed position, in which the mutually opposing inner sides of the arms are brought permanently closer to one another.

In both open and minimally invasive surgery the application of ligature clips for secure and rapid closure of blood vessels and other hollow organs, such as for example bile ducts, is regarded as a tried and tested procedure. The ligation of vessels and hollow organs is necessary if tissue parts are to be resected and if, when cutting through tissue, bleeding occurs that may only with difficulty or not safely be brought under control by means of coagulation.

For this purpose, predominantly clips made of pure titanium are used. This is a proven material that has excellent biocompatibility properties. However, clips made of absorbable materials are also known, which are mostly clips made of polylactates or clips made of other plastics materials, for example polyetheretherketone.

Ligature clips of this type are for example sold by the company AESCULAP AG & Co. KG under the brand name Challenger Ti (Leaflet: Titanium ligature clips and application forceps of the company AESCULAP AG & Co. KG, C 461 11, date of publication February 2002).

Known clips of this type are often of a V-shaped design, the width of the clips varying dependent upon size between 0.5 and 1.2 millimetres. The inner surfaces as a rule are provided with profiling in order to achieve a better seat on the hollow organ in longitudinal direction.

Despite this configuration, with known clips there is the risk that they may slide off the clipped hollow organ. Usually, after a ligature clip has been applied, the hollow organs are transected closely adjacent to the clip and what may then happen is that the clip is unintentionally pushed down laterally of the hollow organ, for example during the removal of abdominal swabs that are positioned over clipped vessels or during minimally invasive surgery by instruments that move out of the range of vision of the surgeon and can therefore be monitored by him only with difficulty.

The object of the invention is to design a ligature clip of the described type in such a way that its seat on the clipped hollow organ is improved.

### SUMMARY OF THE INVENTION

In a ligature clip of the initially described type, this object is achieved according to the invention in that it comprises an endless web member, which in the region of the two arms and the connection point forms two juxtaposed portions that merge one into the other at the free ends of the arms remote from the connection point.

What is then obtained is a double clip having two juxtaposed arm parts, this double configuration of the arm parts markedly improving the application of the ligature clip on the hollow organ. Such a ligature clip might also be regarded as a constructional unit comprising two juxtaposed ligature clips, which are connected to one another in the region of the free ends of the arms by a bridge. The two parts of the clip therefore stabilize one another, with the result that the retaining properties of the ligature clip applied on the hollow organ are so good that it is generally possible to forego the previous practice of placing two such ligature clips independently of one another side by side on a hollow organ remaining in the body.

It is advantageous if the web member has a constant width and/or height over the entire length. In particular, the web member may have a constant cross section over its entire length. This cross section may be for example rectangular.

In a preferred form of construction it is provided that the juxtaposed portions are of a rectilinear design and in particular extend parallel to one another.

It is particularly advantageous if the juxtaposed portions of an arm merge in an arcuate manner into one another in the region of the free end of the arm. What is then obtained is an atraumatic configuration in the region of the free end of the arms, so that even in the event of unsighted application there is no danger of the free ends accidentally damaging the surrounding tissue during closing of the clips. By virtue of the double configuration of the ligature clip, the ligature clip is generally of a wider construction in the region of the free end of the arms, and the arcuate design, which may in particular be circular-arcuate, additionally helps to prevent damage.

In a further preferred form of construction of the invention it is provided that the juxtaposed portions of the arms at their end remote from the connection point open into transverse portions, which project laterally in opposite directions and extend substantially transversely of the longitudinal direction of the juxtaposed portions. This results in an, as a whole, T-shaped configuration of the arms, wherein the transverse portions may for example form clamping jaws.

It is further advantageous if the transverse portions of an arm at their outer ends remote from the juxtaposed portions merge into a clamping portion, which extends at a distance from the transverse portions at the side thereof remote from the juxtaposed portions. The transverse portions and the clamping portion connecting them therefore form at the free end of the arms a clamping surface that projects in both directions from the arms. Between the clamping portion, on the one hand, and the transverse portions, on the other hand, there is a space similar to that between the two juxtaposed portions of the arms, so that tissue clamped in this region may penetrate into the space. This leads to a particularly secure application of the clamping surface formed by the clamping portion and the two transverse portions.

In particular, it may be provided that the clamping portion extends substantially parallel to the transverse portions.

The transverse portions may merge at the outer ends in an arcuate manner, in particular in a circular-arcuate manner, into the clamping portion. The result is therefore an atraumatic configuration of the clamping surfaces in the region of the outer ends.

Preferably, the transverse portions and optionally the clamping portion lie in the same plane as the juxtaposed portions adjoining the transverse portions.

The clamping portion may be of a rectilinear design but, according to a particularly preferred form of construction, it extends in an arcuate manner, in particular in a circular arcuate manner.

In this case, a particularly advantageous configuration arises if the clamping portion is bent in a direction away from the juxtaposed portions, i.e. if the middle of the clamping portion lies closer to the juxtaposed portions than the outer ends. Such a clip may be applied particularly advantageously onto the outside of a hollow organ, as the arcuate shape of the clamping portion may follow the curved outer shape of the hollow organ.

The ligature clips are preferably manufactured with clamping portions of differing curvature, i.e. in a set there is a plurality of ligature clips of differing curvature, thereby allowing the use of clips adapted to the respective dimensions of the hollow organs.

According to a preferred form of construction it is provided that at least one clamping portion carries a projection that, when the clamping portions of the two arms lie adjacent to one another, engages into a recess of the other clamping portion. Thus, on the one hand the clamping portions are guided relative to one another while they are being brought closer together, on the other hand the projections, which may in particular take the form of a pin, penetrate the tissue held between the clamping portions and consequently fix tissue and ligature clip relative to one another.

A plurality of projections may be arranged on, and distributed over the longitudinal direction of, the clamping portion of one arm and corresponding recesses may be disposed on the clamping portion of the other arm, so that a fixing of the tissue is effected over the entire length of the clamping portion.

In a modified embodiment it may also be provided that the juxtaposed portions extend in an arcuate manner. Consequently, the width of the arms varies over their length. In particular, a construction is advantageous, in which the juxtaposed portions extend in a circular arcuate manner. This circular arc contour may extend to such an extent that the juxtaposed portions in the region of the free end of the arms merge with a constant radius of curvature one into the other. Very wide clips are therefore obtained, which for example are suitable for application on the dura.

It is advantageous if the two juxtaposed portions of an arm at least over part of their length lie in one plane. In particular, it may be provided that the two juxtaposed portions of an arm in each case over part of their length lie in two different planes and these planes are inclined towards one another. During the application of such clips a deformation of the clip then occurs not only in the region of the connection point of the two arms but optionally also in the region of the line of intersection of these two planes, the arms in this region prior to closing having a kink or bend and in the course of closing being deformed in this region in such a way that the arms are straightened.

It is advantageous if the web member is profiled at its side forming the inner side of the arms. In this way a more secure seat on a hollow organ arises.

In particular, the profiling in the region of the free end of the arms may be mouse-tooth profiling, i.e. comprise intermeshing teeth. The hollow organ gripped between the arms is thereby reliably prevented from sliding out of the gap between the arms.

In a further preferred form of construction it is provided that the arms at their free end carry a detent device that, when the arms are brought closer to one another, leads to a detent connection of the arms in this region. This too ensures that the gripped hollow organ is unable to slide laterally out of the space between the arms.

It is advantageous if the detent device comprises at least one projection on one arm that in the closed state of the ligature clip engages behind part of the other arm.

For example, the projection on its free end may have a widened portion or a bent portion or take the form of a hook.

It is particularly advantageous if the web member is an integral component. For example, the web member may be an endless band made of titanium or a titanium alloy, this endless band is bent by folding around a centre line, the web member in the region of this centre line then forms the connection point of the ligature clip. In this case, one may start with a circular band and then obtain a relatively wide ligature clip having portions of the arms bent in the shape of a circular arc, or alternatively with a band having parallel, rectilinear portions, which at their ends are connected in an arcuate manner to one another. What is obtained in the latter case is a clip having parallel, rectilinear portions of the web member in the arms with a space between the web members in the form of a parallel longitudinal slot.

In a preferred form of construction of the invention it may be provided that the two juxtaposed portions are connected to one another in the region of the connection point by means of a web. This increases the stability of the ligature clip, in particular preventing the spacing of the two juxtaposed portions from varying during the deformation of the ligature clip.

The configuration of the ligature clip comprising two juxtaposed web portions in the arms and in the region of the connection point also results in this ligature clip in a particularly advantageous manner being able to be accommodated in a magazine, in which a larger number of ligature clips are held in readiness for removal by an appropriate application instrument.

The invention accordingly also relates to a set of a plurality of ligature clips of the previously described type and is characterized in that the ligature clips are disposed on a common carrier that passes through the ligature clips, which are arranged parallel and in a row adjacent to one another, in the region of the space between the juxtaposed portions. By virtue of the ligature clips having a space surrounded by the portions of the web member, this space may be utilized to push a carrier through it so that a larger number of identically constructed ligature clips may be accommodated parallel and adjacent to one another on the carrier.

In this case, it is particularly advantageous if the juxtaposed portions lie at least in places closely adjacent to the carrier and therefore lead to friction locking between the ligature clips and the carrier, so that the ligature clips are locked against unintended displacement on the carrier. To remove a clip, the carrier has to be displaced with a specific force relative to the ligature clip in order to overcome this force-locking between ligature clip and carrier.

The following description of preferred forms of construction of the invention is used in connection with the drawings to provide a detailed explanation.

### BRIEF DESCRIPTION OF THE DRAWINGS

FIG. 1: a ligature clip having two juxtaposed web member portions in open position;

FIG. 2: a partial view of an application instrument with a ligature clip according to FIG. 1 in open position of the ligature clip;

FIG. 3: a perspective partial view of the clamping jaw of a modified embodiment of an application instrument without ligature clip;

FIG. 4: a view similar to FIG. 3 with a ligature clip inserted between both clamping jaws prior to closing of the ligature clip;

FIG. 5: a ligature clip according to FIG. 1 applied on a hollow organ and in closed position;

FIG. 6: a view similar to FIG. 1 of a modified embodiment of a ligature clip with mouse-tooth profiling on the free ends of the two arms;

FIG. 7: a view similar to FIG. 5 of a modified embodiment with further mouse-tooth profiling;

FIG. 8: a view of a ligature clip similar to FIG. 1 having a detent projection on the free end of one arm;

FIG. 9: a partial view of a modified embodiment of such a detent projection prior to engagement into a detent recess of the other arm of the ligature clip;

FIG. 10: a view similar to FIG. 7 having a hook-shaped detent element on one arm of the ligature clip;

FIG. 11a and FIG. 11b: a plan view and side view respectively of a band-shaped material prior to bending into a ligature clip;

FIG. 12: a side view of the ligature clip of FIG. 9 in the open state;

FIG. 13: a side view of the ligature clip of FIG. 12 in the partially closed state;

FIG. 14: a side view of the ligature clip of FIG. 12 in the closed state;

FIG. 15: a perspective view of a carrier having a larger number of ligature clips according to FIG. 1;

FIG. 16: a perspective view of a further preferred embodiment of a ligature clip with a T-shaped design of the arms in the open state;

FIG. 17: a schematic view of two hollow organs that are connected to one another by means of ligature clips according to FIG. 16 and

FIG. 18: a sectional view through the application region of two hollow organs along line 18-18 in FIG. 17.

### DETAILED DESCRIPTION OF THE INVENTION

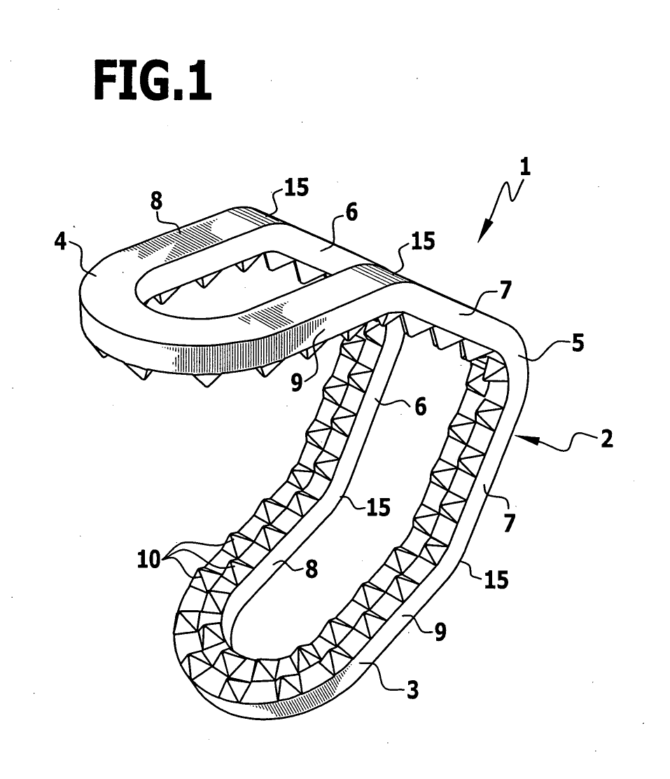

The ligature clip 1 shown in FIGS. 1 to 5 comprises an endless strip-shaped band 2 of a permanently flexible material, for example titanium, a titanium alloy or one of the plastics materials conventionally used to manufacture such ligature clips. It is of course also possible to use other materials, for example steels, provided that the materials used are biocompatible. In the illustrated embodiment, the band 2 has a rectangular cross section, which remains constant over the entire length of the band 2 and in which the width of the band is approximately twice as great as the height of the band.

The endless band 2 in this case is disposed in such a way that it forms over a large part of its length two rectilinear regions, which run parallel at a spacing adjacent to one another and which at their ends merge one into the other by virtue of a circular arc shape of the band. Along a centre line extending transversely of the longitudinal extent of the rectilinear regions the two halves of the endless band 2 are folded on top of one another, thereby resulting in two mutually opposite arms 3, 4, which in the region of the said centre line form a connection point 5 deformed in a substantially arcuate manner. From this connection point 5 the two parallel juxtaposed parts of the band 2 extend in the form of rectilinear portions 6, 7 in one plane and are adjoined by further rectilinear portions 8, 9, which likewise lie in one plane, the two planes being inclined approximately by an angle of 30° towards one another. The two portions 8, 9 in the region of the free end of the arms 3, 4 merge into one other in the shape of a circular arc extending over 180°.

Each of the arms 3, 4 at the inner side facing the respective other arm carries profiling in the form of tapering teeth 10 that project in the direction of the other arm, this profiling extending over the entire length of the band 2.

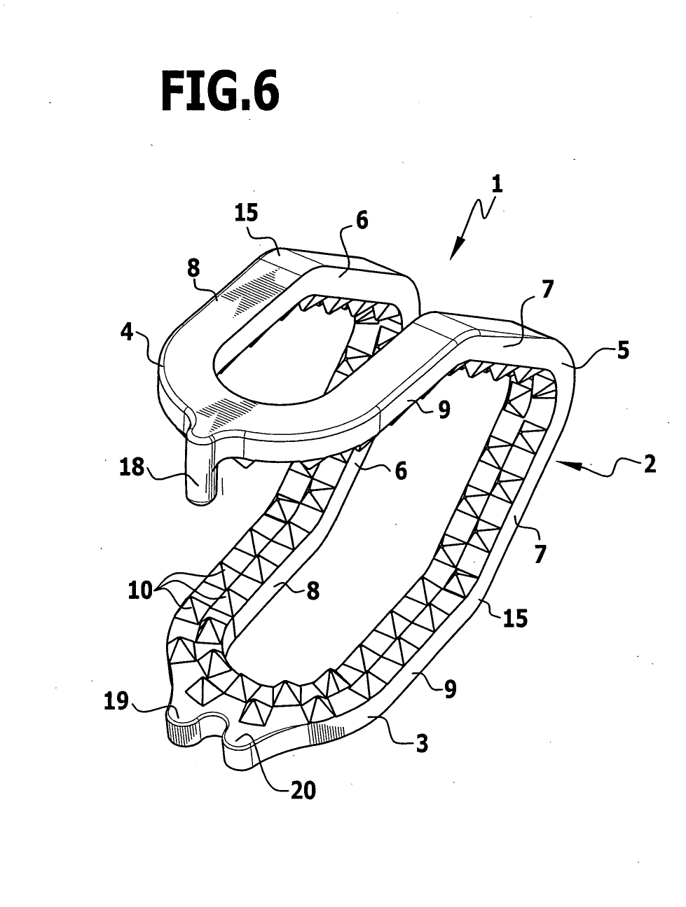

In FIG. 6 a ligature clip 1 is shown, which corresponds substantially to that of FIG. 1, for which reason mutually corresponding parts bear the same reference characters. Unlike the ligature clip of FIG. 1, the two arms 3, 4 of the ligature clip 1 carry on their free ends additional projections 18, 19, 20, which are so disposed that in the closed state of the ligature clip the projection 18 on one of the two arms engages between the projections 19, 20 on the other arm and therefore, on the one hand, closes off the space between the two arms 3, 4 in a forward direction and, on the other hand, locks the two arms against lateral displacement relative to one another.

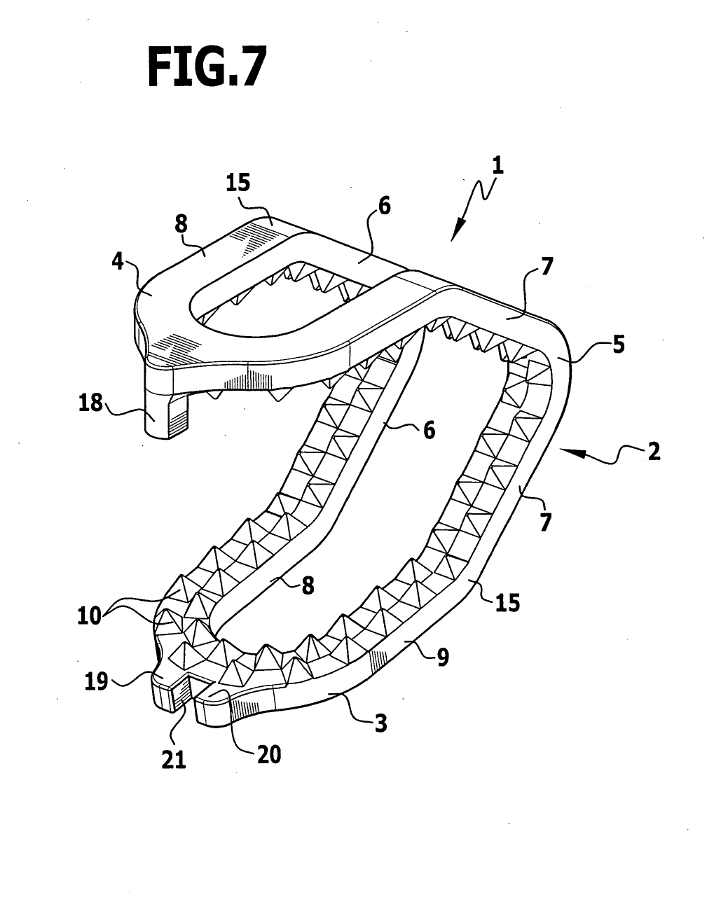

For the ligature clip of FIG. 7 a very similar configuration has been selected, the projections 18, 19, 20 in this case being even more ingenious so that the projection 18 on one arm engages positively into a recess 21 between the projections 19, 20 on the other arm. Such a fit is also described as mouse-tooth profiling.

FIG. 8 shows a similar ligature clip 1, in which the two arms in the closed state may be interlocked. For this purpose, one of the two arms carries on its free end a detent projection 22, which is bent laterally at its free end. In the closed state the lateral bend 23 of the detent projection 22 may slide past and engage behind the other arm.

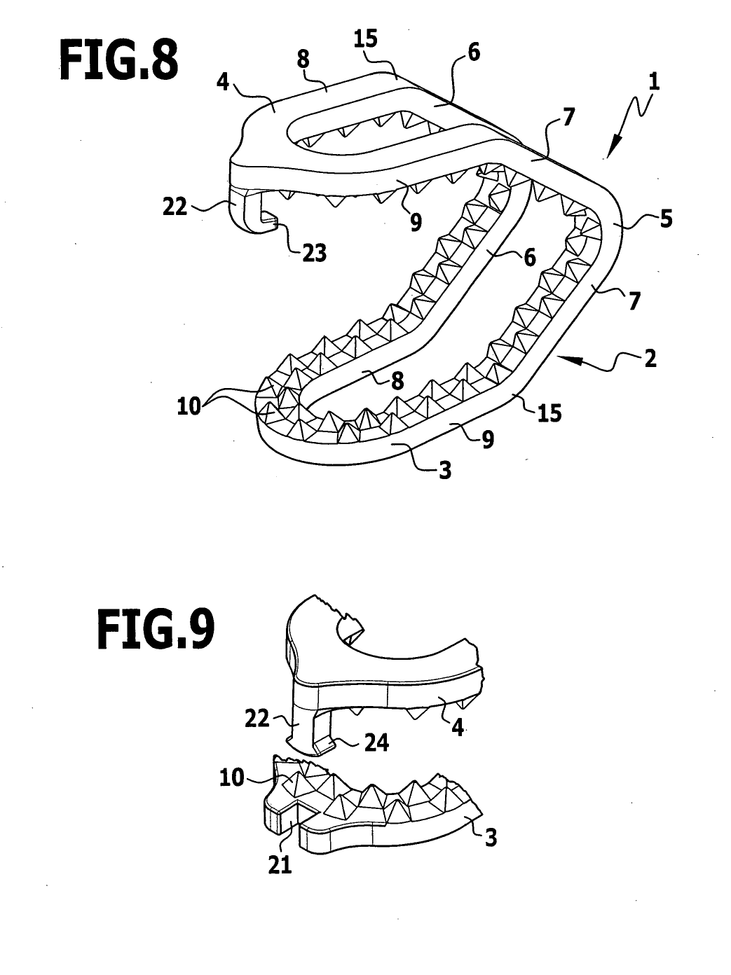

In FIG. 9 a modified design of a detent device is shown, where the detent projection 22 is laterally thickened at its free end. In the closed state of the ligature clip, the detent projection 22 engages into a recess 21 of the other arm, and the thickened portion 24 on the free end of the detent projection 22 engages behind the other arm. This detent device simultaneously performs a similar function to mouse-tooth profiling, the two arms namely also being additionally locked against lateral displacement relative to one another.

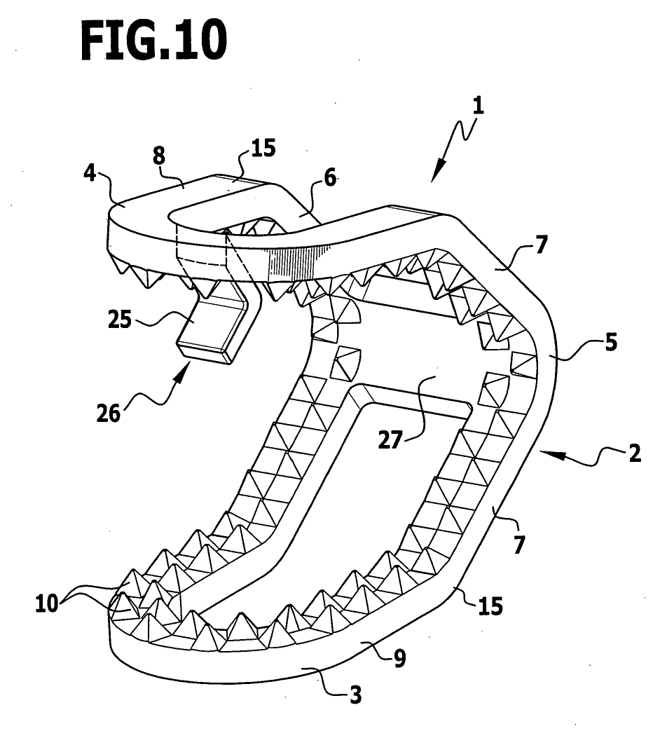

In the ligature clip shown in FIG. 10, one of the two arms carries a tongue 25, which projects from the free end into the gap between the juxtaposed portions and which approximately in the middle of its length is bent through almost 180° and hence forms a detent hook 26, which in the closed state of the ligature clip engages into the gap between the juxtaposed portions of the other arm and engages behind this arm. In this way, both arms are likewise interlocked in the closed state.

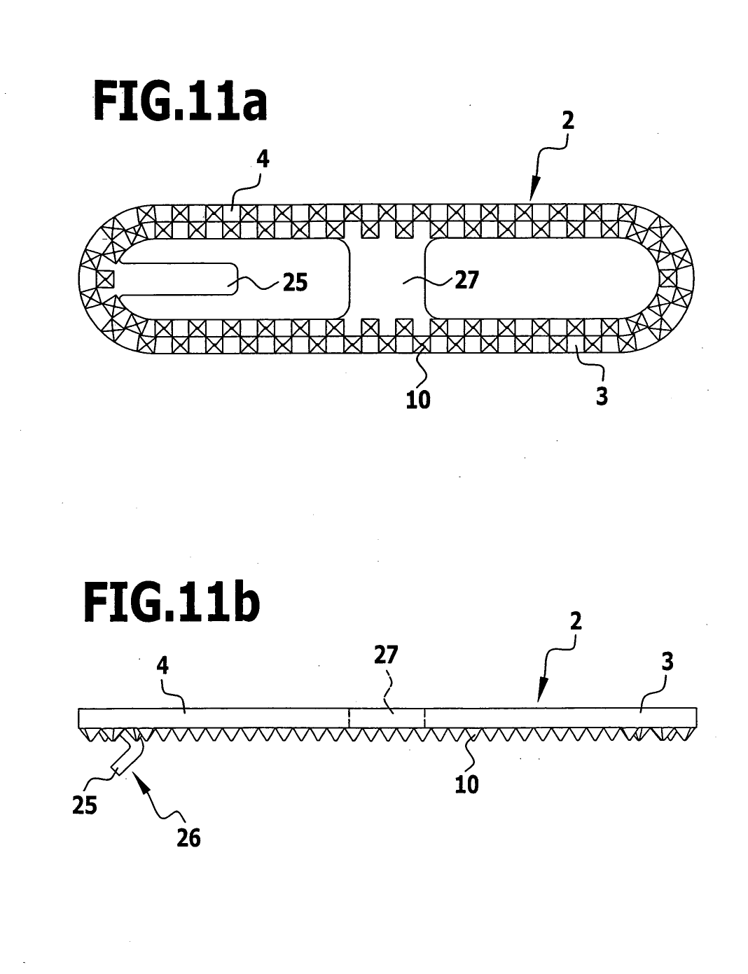

The starting point for manufacturing the ligature clip 1 is an endless band, which lies in one plane and is preformed with rectilinear portions and circular-arcuate connection regions. FIGS. 11a, 11b and 12 to 14 illustrate this manufacturing operation using as an example the ligature clip of FIG. 10. The initial component in the form of an endless band is bent firstly around the centre line and secondly in the region between the portions 6 and 8 and/or 7 and 9 in such a way that the two planes defined in each case by these portions are inclined towards one another. The portions 6 and/or 7 of the two arms 3, 4 immediately adjoining the connection point 5 form an opening angle of approximately 60°, this position being described as the open position of the ligature clip 1. In this position of the arms 3 the ligature clip 1 is approximately V-shaped in cross section and capable of being applied with the aid of an application instrument 11 laterally onto a hollow organ 12, to which it is to be fixed.

The ligature clip of FIGS. 10, 11a and 11b has in the region of the connection point 5 a transverse web 27, by means of which the stability of the ligature clip is increased, but this transverse web is not absolutely necessary and the embodiments of FIGS. 1, 6, 7 and 8 have no such transverse web.

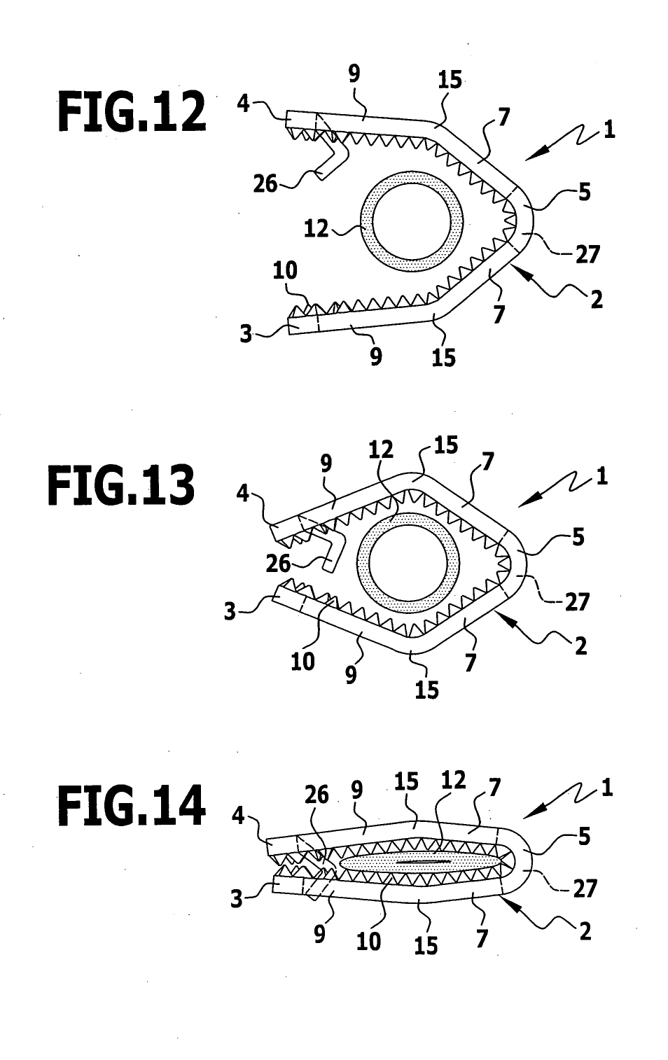

FIGS. 12, 13 and 14 show the shape of the ligature clip during application onto a hollow organ 12, during the closing operation and at the end of the closing operation. It is clear that in the closed state of the ligature clip the detent hook 26 engages behind the opposite arm at the free end thereof and hence closes the ligature clip in the region of the free ends of the arms and moreover locks the two arms against lateral displacement.

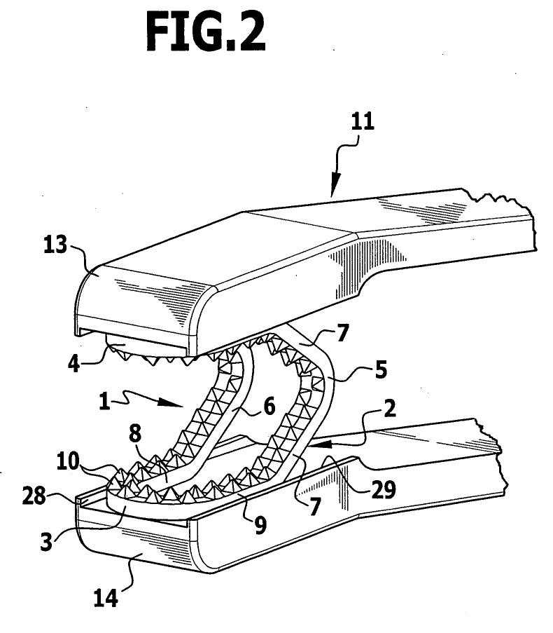

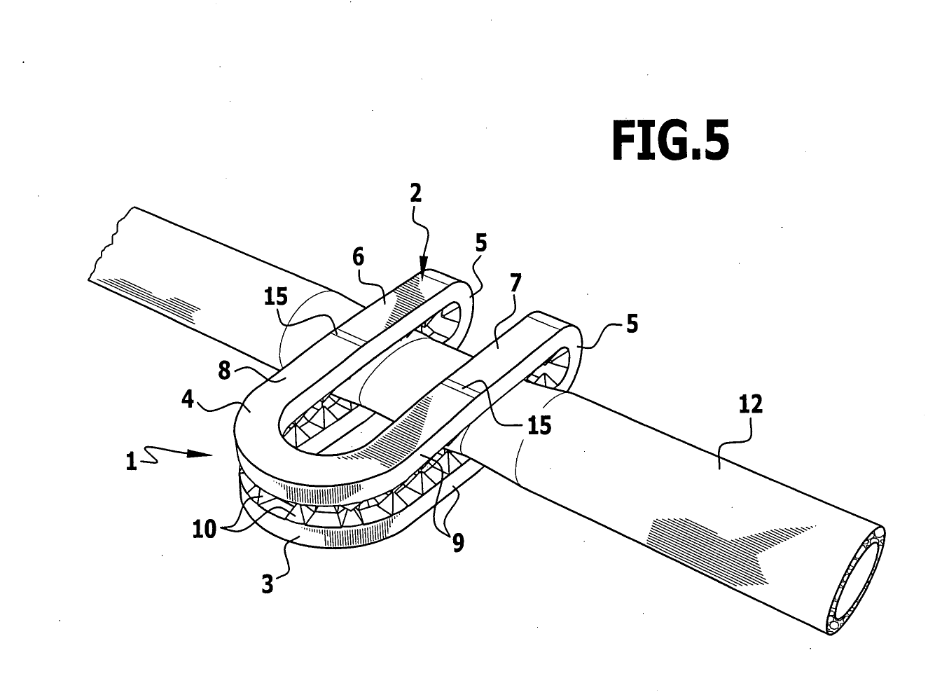

In FIG. 2 an application instrument 11 with an inserted ligature clip 1 in open position is schematically shown. The application instrument 11 in this case comprises two clamping jaws 13, 14, which may be applied onto the outside of the two arms 3, 4 and brought closer to one another by means of a mechanism not shown in the drawing, with the result that the two arms 3, 4 disposed at opposite sides of a hollow organ 12 are pressed towards one another. In this case, the ligature clip 1 is deformed in the region of the connection point 5 and optionally also in the region of the bend 15 of the arms 3, 4 between the portions 6 and 8 and/or 7 and 9, so that the arms 3, 4 are then applied against one another, thereby firmly enclosing the hollow organ 12 between them, and are substantially straight, as is shown in FIG. 5. The arms 3, 4 in this case comprise two juxtaposed web members, i.e. take the form of a double clip, and lie in two juxtaposed regions against the hollow organ, which is therefore gripped securely by the ligature clip 1.

In the embodiment of FIG. 2, the clamping jaws 13, 14 of the application instrument 11 are constructed slightly wider than the ligature clip 1, the clamping jaws 13, 14 for centring of the ligature clip 1 carrying on their outer edges laterally upward-projecting shoulders 28, 29, which rest against the outer sides of the portions 6, 7, 8, 9 and hence ensure that the ligature clip is securely positioned between the clamping jaws.

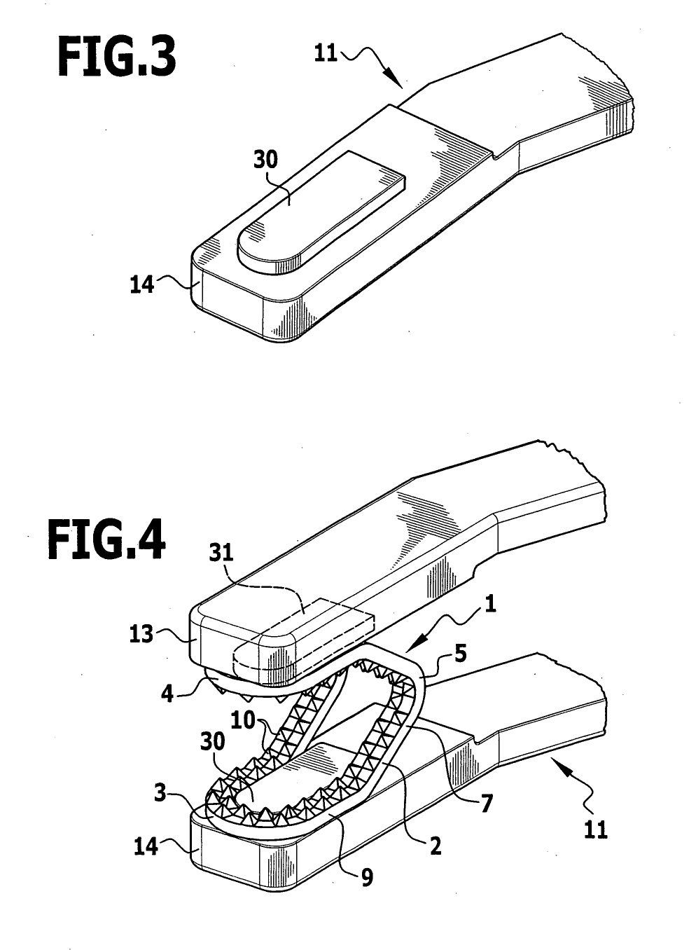

FIGS. 3 and 4 show a further embodiment of such an application instrument 11, in which the clamping jaws 13, 14 carry centring projections 30, 31 that fit precisely into the space between the portions 6, 7 and 8, 9 of the arms 3, 4 once the clamping jaws 13, 14 have been applied onto the outer sides of the arms 3, 4. This likewise results in a precise and stable positioning of the ligature clip between the clamping jaws 13, 14, which in this embodiment may optionally also be constructed narrower than the ligature clips 1.

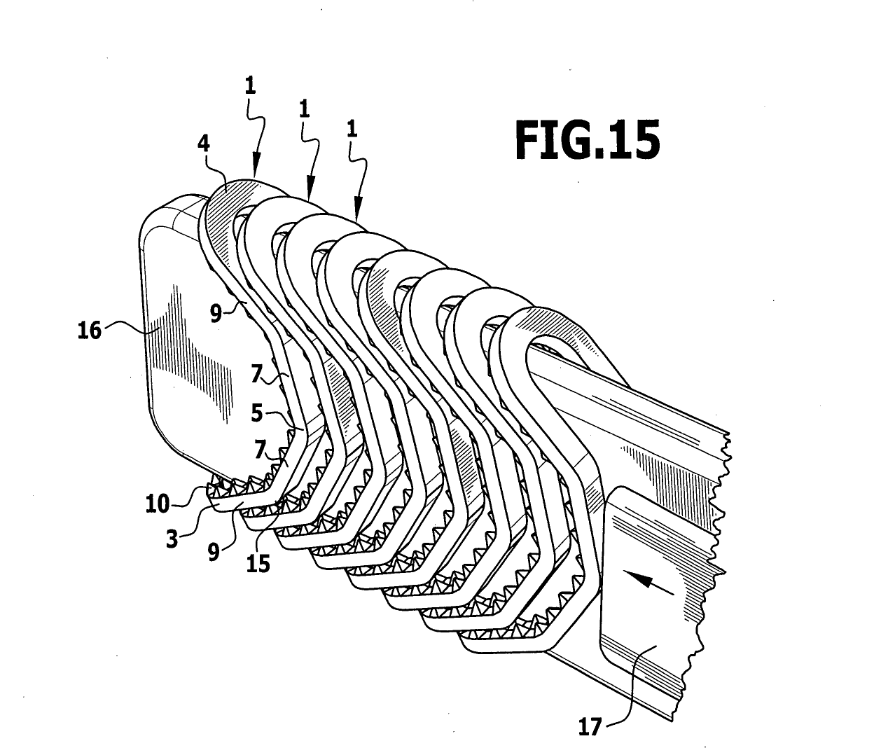

In FIG. 15 a carrier 16 is schematically shown, which is rectangular in cross section and onto which a fairly large number of ligature clips 1 of the type represented in FIGS. 1 to 3 have been slipped in open position. The dimensions of the carrier 16 are in this case so selected that its width corresponds to the width of the space between the juxtaposed web members of the arms 3, 4, so that the portions 6, 7, 8, 9 of the arms 3, 4 lie closely adjacent to the carrier 16 and therefore fix the ligature clips 1 non-positively on the carrier 16. All of the ligature clips 1 are in this case disposed parallel and immediately adjacent to one another so that in a very confined space a fairly large number of such ligature clips 1 may be stored on the carrier 16. By means of an ejector element 17, which is shown only schematically in the drawing, the ligature clips 1 may be displaced along the carrier 16 in such a way that in each case the foremost ligature clip 1 may be released for application. Such a release may alternatively be effected by other means, for example by means of an application instrument 11 that grasps in each case only the foremost ligature clip or only the rearmost ligature clip from the supply of ligature clips 1 on the carrier 16 and may remove it from the carrier. The essential point is that by configuring the ligature clips 1 with two juxtaposed web members there is formed between the web members a location space for the carrier that without further resources allows the ligature clips 1 to be stored on a carrier slid into this location space.

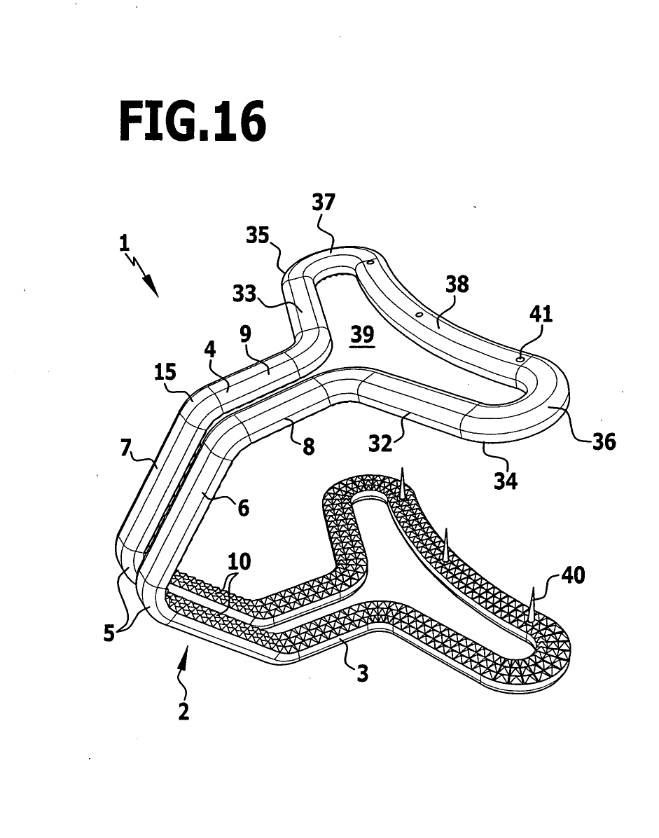

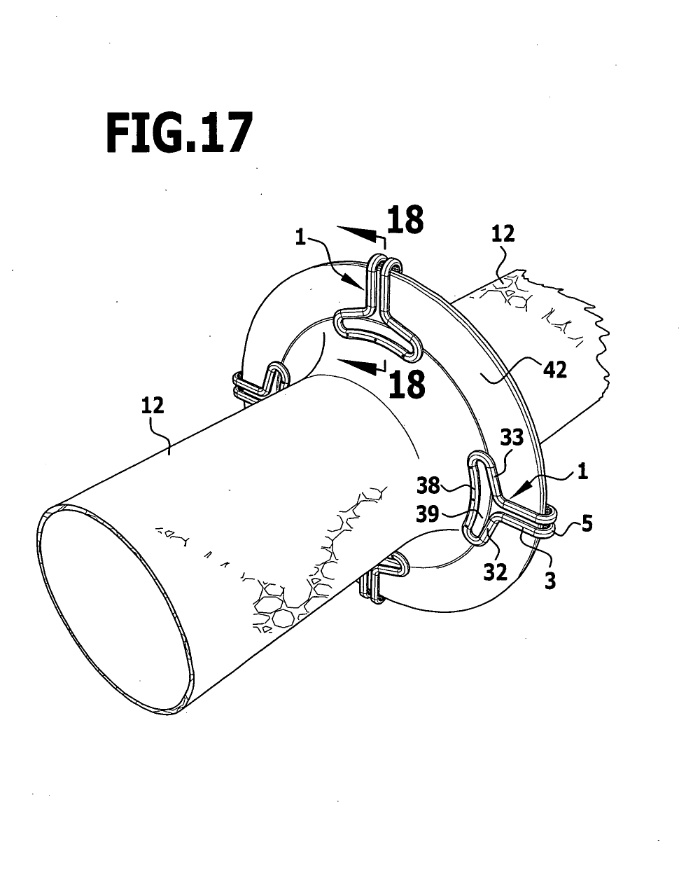

In FIGS. 16 to 18 a modified embodiment of a ligature clip 1 is described. This is of a similar construction to the ligature clip of the previously described embodiments, for which reason mutually corresponding parts bear the same reference characters.

In the ligature clip of FIGS. 16 to 18 the juxtaposed portions 8 and 9 on the free end of the arms 3, 4 do not merge directly into one another in the shape of an arc, rather the free ends of the portions 8 and 9 are adjoined by transverse portions 32, 33, which project outwards and hence in opposite directions and extend in the same plane as the juxtaposed portions 8 and 9. At their outer ends 34, 35 remote from the portions 8 and 9, the transverse portions 32, 33 run via circular-arcuate end portions 36, 37 extending approximately over 180° into a common clamping portion 38, which extends at the side of the transverse portions 32, 33 remote from the juxtaposed portions 8, 9 with a spacing relative to the transverse portions 32, 33 and connects the two end portions 36, 37 to one another. The clamping portion 38 in the embodiment represented in FIGS. 16 to 18 extends between the end portions 36 and 37 in the shape of a circular arc, wherein the outer ends 34, are bent outwards, i.e. in a direction away from the arms 3, 4. The clamping portion 38 lies in the same plane as the transverse portions 32, 33 and the arcuate end portions 36, 37, with the transverse portions 32, 33, the end portions 36, 37 and the clamping portion 38 forming a common clamping surface on the free end of the arms 3, 4.

In the embodiment of FIG. 16 the transverse portions 32, 33 project at an angle of approximately 45° from the juxtaposed portions 8, 9, however this angle may alternatively be an angle in the order of magnitude of 90°. In particular, the transverse portions 32, 33 and the clamping portion 38 may extend parallel to one another. In any case, a space 39 remains between the transverse portions 32, 33 and the clamping portion 38.

The ligature clip of FIGS. 16 to 18 is likewise formed integrally from a band 2 and carries on its inner side teeth 10 or similar profiling.

There is moreover disposed on, and distributed over the length of, one of the two clamping portions 38 a plurality of pin-shaped projections 40, opposite each of which is a bore 41 in the other clamping portion, into which the pin-shaped projection 40 engages when the two clamping portions 38 of the two arms 3, 4 are brought close to one another.

The ligature clip shown in FIGS. 16 to 18 is particularly suitable for connecting two hollow organs 12. At the mutually abutting ends of these hollow organs their edge 42 is bent laterally outwards so as to produce a flange-shaped projection. In this region a plurality of ligature clips may be disposed side by side in peripheral direction in such a way that the arcuate clamping portions 38 follow the external contour of the hollow organs 12.

In this case, it is particularly advantageous if ligature clips are used, in which the curvature of the clamping portions 38 corresponds to the curvature of the hollow organs. For this purpose, in a set the surgeon has at his disposal ligature clips having clamping portions 38 of differing curvature, thereby allowing him to select from this set the ligature clips that are in each case adapted to the curvature of the hollow organ.

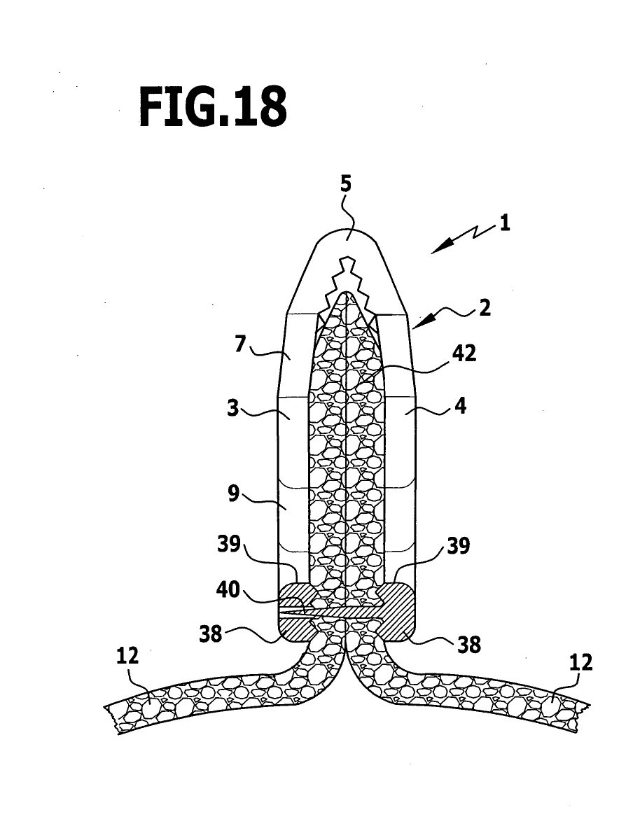

Once the ligature clips have been applied and closed in such a way that the clamping portions 38 of the two arms 3, 4 are pressed against one another, the clamping portions 38 and optionally also the transverse portions 32, 33 accommodate between them the tissue, i.e. in the example of the connection of the hollow organs 12 the edge 42, as is shown in FIG. 18. The pin-shaped projections 40 in this case penetrate the edge 42 and engage into the bore 41 of the opposite clamping portion 38, thereby ensuring a secure fixing of the ligature clips to the edge 42. This secure fixing is also assisted by the fact that the tissue of the edge 42 enters the gap 39 between the clamping portion 38, on the one hand, and the two transverse portions 32, 33 and hence prevents a lateral displacement of the ligature clip relative to the edge.

1. Surgical ligature clip comprising two retaining arms, which are connected to one another in each case at one end by a deformable connection point and bendable in such a way towards one another that the arms move from an open position, in which they are spaced further apart from one another, into a closed position, in which the mutually opposing inner sides of the arms are brought permanently closer to one another, wherein it comprises an endless web member, which in the region of the two arms and the connection point forms two juxtaposed portions that merge into one other at the free ends of the arms remote from the connection point.

2. Ligature clip according to claim 1, wherein the web member has a constant width over its entire length.

3. Ligature clip according to claim 1, wherein the web member has a constant height over its entire length.

4. Ligature clip according to claim 1, wherein the web member has a constant cross section over its entire length.

5. Ligature clip according to claim 1, wherein the web member has a rectangular cross section over its entire length.

6. Ligature clip according to claim 1, wherein the juxtaposed portions are of a rectilinear form.

7. Ligature clip according to claim 6, wherein the juxtaposed portions extend parallel to one another.

8. Ligature clip according to claim 1, wherein the juxtaposed portions of an arm merge in an arcuate manner into one another in the region of the free end of the arm.

9. Ligature clip according to claim 8, wherein the juxtaposed portions of an arm merge in a circular-arcuate manner into one another in the region of the free end of the arm.

10. Ligature clip according to claim 1, wherein the juxtaposed portions at their end remote from the connection point open into transverse portions that project laterally in opposite directions.

11. Ligature clip according to claim 10, wherein the transverse portions at their outer ends remote from the juxtaposed portions merge into a clamping portion that extends at a spacing from the transverse portions at the side thereof remote from the juxtaposed portions.

12. Ligature clip according to claim 11, wherein the clamping portion extends substantially parallel to the transverse portions.

13. Ligature clip according to claim 11, wherein the transverse portions at the outer ends merge in an arcuate manner into the clamping portion.

14. Ligature clip according to claim 13, wherein the transverse portions at the outer ends merge in a circular-arcuate manner into the clamping portion.

15. Ligature clip according to claim 10, wherein the transverse portions and optionally the clamping portion lie in the same plane as the juxtaposed portions adjoining the transverse portions.

16. Ligature clip according to claim 11, wherein the clamping portion extends in an arcuate manner.

17. Ligature clip according to claim 16, wherein the clamping portion extends in a circular arcuate manner.

18. Ligature clip according to claim 16, wherein the clamping portion is bent in a direction away from the juxtaposed portions.

19. Ligature clip according to claim 11, wherein at least one clamping portion carries a projection that, when the clamping portions of the two arms lie adjacent to one another, engages into a recess of the other clamping portion.

20. Ligature clip according to claim 19, wherein the projection is a pin.

21. Ligature clip according to claim 19, wherein a plurality of projections are disposed on, and distributed over the longitudinal direction of, the clamping portion of one arm and corresponding recesses are disposed on the clamping portion of the other arm.

22. Ligature clip according to claim 1, wherein the juxtaposed portions extend in an arcuate manner.

23. Ligature clip according to claim 22, wherein the juxtaposed portions extend in a circular arcuate manner.

24. Ligature clip according to claim 23, wherein the juxtaposed portions in the region of the free end of the arms merge into one another with a constant radius of curvature.

25. Ligature clip according to claim 1, wherein the two juxtaposed portions of an arm at least over part of their length lie in one plane.

26. Ligature clip according to claim 25, wherein the two juxtaposed portions of an arm in each case at least over part of their length lie in different planes and that the two planes are inclined towards one another.

27. Ligature clip according to claim 1, wherein the web member is profiled on its side forming the inner side of the arms.

28. Ligature clip according to claim 27, wherein the profiling in the region of the free end of the arms is mouse-tooth profiling.

29. Ligature clip according to claim 1, wherein the arms on their free ends carry a detent device that, when the arms are brought closer to one another, leads to a detent connection of the arms in this region.

30. Ligature clip according to claim 29, wherein the detent device comprises at least one projection on one arm that in the closed state of the ligature clip engages behind a part of the other arm.

31. Ligature clip according to claim 30, wherein the projection has on its free end a widened portion or a bent portion.

32. Ligature clip according to claim 30, wherein the projection takes the form of a hook.

33. Ligature clip according to claim 1, wherein the web member is an integral component.

34. Ligature clip according to claim 1, wherein the two juxtaposed portions are connected to one another in the region of the connection point by means of a web.

35. Ligature clip according to claim 10, wherein the two juxtaposed portions are connected to one another in the region of the connection point by means of a web.

36. Set of a plurality of ligature clips, each of which comprises two retaining arms, which are connected to one another in each case at one end by a deformable connection point and bendable in such a way towards one another that the arms move from an open position, in which they are spaced further apart from one another, into a closed position, in which the mutually opposing inner sides of the arms are brought permanently closer to one another, each ligature clip comprising an endless web member that forms in the region of the two arms and the connection point two juxtaposed portions that merge one into the other at the free ends of the arms remote from the connection point, wherein all of the ligature clips are disposed on a common carrier that passes through the ligature clips, which are disposed parallel and in a row adjacent to one another, in the region of the space between the juxtaposed portions.

37. Set of a plurality of ligature clips according to claim 36, wherein the juxtaposed portions of the ligature clips lie at least in places closely adjacent to the carrier.
